(19) United States
(12) Patent Application Publication
(10) Pub. No.: US 2008/0051315 A1
(43) Pub. Date: Feb. 28, 2008

(54) Grp Receptor-Related Methods for the Treating and Preventing Fear-Related Disorders

(76) Inventors: Eric Kandel, Riverdale, NY (US); Gleb Shumyatsky, Tenafly, NJ (US); Vadim Bolshakov, Waltham, MA (US); Gael Malleret, New York, NY (US)

Correspondence Address: COOPER & DUNHAM, LLP, 1185 AVENUE OF THE AMERICAS, NEW YORK, NY, 10036, US

(21) Appl. No.: 10/582,303

(22) PCT Filed: Dec. 9, 2004
(86) PCT No.: PCT/US04/41388

§ 371 Date: May 11, 2007

Related U.S. Application Data
(60) Provisional application No. 60/529,449, filed on Dec. 11, 2003.

(51) Int. Cl. C07H 21/04; A61P 43/00; C07H 19/00

(52) U.S. Cl. 514/1; 536/22.1; 536/23.1

(57) ABSTRACT

This invention provides a method for treating a subject afflicted with a fear-related disorder comprising administering to the subject a therapeutically effective amount of a gastrin-releasing peptide receptor agonist. This invention further provides a method for inhibiting in a subject the onset of a fear-related disorder resulting from exposure to a traumatic experience comprising administering a prophylactically effective amount of a gastrin-releasing peptide receptor agonist to the subject prior to and/or following the traumatic experience.

The invention disclosed herein was made with U.S. government support under grant number MH50733 from the National Institute of Mental Health and with support under grant numbers NS44185 and DA15098 from the National Institutes of Health. Accordingly, the U.S. government has certain rights in this invention.

Throughout this application, various publications are referenced by author and date. Full citations for these publications may be found at the end of the specification immediately preceding the claims. The disclosures of these references in their entireties are hereby incorporated by reference into this application to describe more fully the art to which this invention pertains.

### BACKGROUND OF THE INVENTION

### Physiology of Fear

Fear is a basic, evolutionally conserved, emotion which triggers a set of defensive mechanisms for adapting to threatening events that is essential for survival. A key component of the neural circuitry of fear, both innate and learned, in humans and in simpler vertebrate experimental animals is the amygdala, a well-defined subcortical nuclear group (Davis and Whalen, 2001; LeDoux, 2000).

The memory of learned fear can be assessed quantitatively using a Pavlovian fear-conditioning paradigm (Fanselow and LeDoux, 1999; Kapp et al., 1992). During fear conditioning, an initially neutral conditioned stimulus (CS) acquires biological significance by becoming associated, following a few pairing trials, with an aversive unconditioned stimulus (US). After learning this association, the animal responds to the previously neutral CS with a set of defensive behavioral response, which includes freezing, increased heart rate, and startle. The CS can be unimodal, involving only a single cure or modality such as a tone, light, smell, or touch. Alternatively, it can be multimodal, involving several sensory modalities such as a context. Unimodal (cued) fear conditioning requires the amygdala but not the hippocampus. By contrast, multimodal (contextual) fear conditioning depends on both the hippocampus and the amygdala.

The lateral nucleus is the input region within the amygdala, where the association of learned information about CS and US occurs during auditory fear conditioning. The sensory information that mediates the CS—the auditory tone—reaches the lateral nucleus by way of two neural pathways, both of which are essential for learned fear (Romanski and LeDoux, 1992). One pathway, the direct thalamo-amygdala pathway, originates in the medial geniculate nucleus (MGm) and in the posterior intralaminar nucleus (PIN) of the thalamus. The second pathway, the indirect cortico-amygdala pathway, extends from the auditory thalamus to the auditory cortex (TE3 area) and includes a further projection that relays the processed auditory information from the cortex to the lateral amygdala. After these two inputs are processed in the lateral nucleus, the signal is distributed to other amygdaloid nuclei (Pitkanen et al. 1997), including the central nucleus of the amygdala (CeA), which projects in turn to areas in the brainstem that control autonomic (heart rate) and somatic motor centers (freezing) involved in the expression of fear.

Anatomical tracing and lesion studies first demonstrated the importance of the lateral nucleus for fear conditioning. Subsequent physiological experiments showed that learning produces prolonged synaptic modification in both of the inputs to the lateral nucleus: the thalamo-amygdala pathway (McKernan and Shinnick-Gallagher, 1997; Rogan et al., 1997) and the cortico-amygdala pathway (Tsvetkov et al., 2002). These synaptic modifications, which accompany behavioral learned fear, are mechanistically similar to LTP induced artificially by electrical stimulation in tissue slices of the amygdala. By providing a direct causal link between slice LTP and memory storage, these studies establish the amygdala as perhaps the simplest and the best model system in the mammalian brain for analyzing the cellular and molecular mechanisms of memory storage.

In contrast to the detailed cellular physiological information that is becoming available, the molecular machinery that underlies synaptic plasticity in amygdala-dependent learned fear is largely unknown.

### Neuropeptides and Anxiolytics

A number of neuropeptides are believed to be involved in the pathophysiology of anxiety, including, for example, cholecytokinin (CCK), corticotropin-releasing factor and neuropeptide Y. Gastrin releasing peptide (GRP) is known as a potent satiety agent (see Merali, Z. et al. 1994). GRP antagonists are also known in the field of cancer research for their use in inhibiting tumor growth.

Gastrin releasing peptide (GRP) and neuromedin B (NMB) are mammalian homologs of bombesin, a 14 amino acid peptide hormone first isolated from the skin of the frog, Bombina bombina. Three bombesin-like peptide receptors are known: gastrin releasing peptide receptor (GRPR; BB2), neuromedin B receptor (NMBR; BB1) and bombesin receptor subtype-3 (BRS-3; BB3). All are G-protein coupled receptors. Gastrin releasing peptide receptor is known in the art by the acronyms GRPR and BB2 (for bombesin recepotr subtype 2). Potent and selective peptide agonists of the gastrin releasing peptide receptor (BB2) are known. For example, Darker, J. G. et al. (2001) and Casibang, M. and Moody, T. W. (2000) describe such agonists. Bombesin agonists are also known, for example Condamine E. et al. (1998).

More generally, assays for agonists or antagonists of G-protein coupled receptors are known in the art. See, for example, Fitzgerald, L. R., (1999). Similarly, one of skill in the art can determine the expression of GRPR in a cell or tissue sample using routine methods (see Kusui et al. 1995).

Gamma-aminobutyric acid (GABA), along with norepinephrine and serotonin, is known to be important in the regulation of anxiety. GABA is the major inhibitory neurotransmitter in the mammalian central nervous system (CNS) and is utilized for intercellular communication by approximately one-third of all synapses in the CNS. There are two classes of GABA receptors, A and B. The GABA-A receptor is comprised of five peptide subunits (alpha, beta, gamma, delta, and rho) which form a chloride-permeable ion channel coupled to a G-protein. Each of the five subunits may have multiple isoforms. For example, there are six alpha, four beta, three gamma, one delta, and two rho subunits known presently.

Anxiolytics are compounds that relieve anxiety. Known anxiolytic compounds include GABA-A agonists such as the benzodiazepines, which are the prototypic anti-anxiety compounds. Benzodiazepines interact with binding sites which are largely defined by the alpha subunit of the GABA-A receptor complex. In older literature, the GABA-A receptor complex was referred to as the “benzodiazepine receptor” or BZR. More than two-dozen benzodiazepines are in clinical use in the United States. Among these are Alprzolam (Xanax), chlordiazepoxide (Librium), and diazepam (Valium). Other examples of anxiolytic compounds are neurohormones such as 3-alpha, 5-alpha-pregnanolone (THPROG) and muscimol.

Animal tests for anxiolytic activity are known in the art. For example, one test involves pairing a reward for which the animal must perform some behavior, such as lever pressing, with an aversive stimulus, such as mild electric shock. Agents that increase the rate of responses punished with the shock tend to be anxiolytic in humans (see basic Neurochemistry, 6th ed. Siegel et al. editors). Another indicator of anxiolytic activity is a compound's binding affinity for the GABA-A receptor.

### SUMMARY OF THE INVENTION

This invention provides a method for treating a subject afflicted with a fear-related disorder comprising administering to the subject a therapeutically effective amount of a gastrin-releasing peptide receptor agonist.

This invention further provides a method for inhibiting in a subject the onset of a fear-related disorder resulting from exposure to a traumatic experience comprising administering a prophylactically effective amount of a gastrin-releasing peptide receptor agonist to the subject prior to and/or following the traumatic experience.

This invention further provides an article of manufacture comprising (a) a packaging material having therein a gastrin-releasing peptide receptor agonist, and (b) a label indicating a use for the agonist in treating, and/or inhibiting the onset of, a fear-related disorder in a subject.

This invention further provides a nucleic acid comprising a gastrin-releasing peptide gene, wherein the gene has inserted into it, either at its start or stop codon, a polypeptide-encoding sequence, wherein the polypeptide is not gastrin-releasing peptide.

This invention further provides a transgenic animal whose somatic cells have stably integrated therein a nucleic acid comprising a gastrin-releasing peptide gene, wherein the gene has inserted into it, either at its start or stop codon, a polypeptide-encoding sequence, wherein the polypeptide is not gastrin-releasing peptide, and wherein the polypeptide is specifically expressed in the animal's amygdala.

Finally, this invention provides a method for producing a transgenic animal whose amygdaloid cells specifically express an exogenous polypeptide, which method comprises producing a transgenic animal by introducing into an oocyte an exogenous DNA so that the exogenous DNA is stably integrated into the oocyte, and permitting the resulting oocyte to mature into a viable animal, wherein (a) the animal's somatic cells have the exogenous DNA stably integrated therein, (b) the exogenous DNA comprises a gastrin-releasing peptide gene, wherein the gene has inserted into it, either at its start or stop codon, an exogenous polypeptide-encoding sequence, and the exogenous polypeptide is not gastrin-releasing peptide, and (c) the exogenous polypeptide is specifically expressed in the animal's amygdala.

### BRIEF DESCRIPTION OF THE FIGURES

FIG. 1: The Grp Gene is Specifically Expressed in the Lateral Nucleus/AB of the Amygdala and in the Cued and Contextual CS Pathways to the Amygdala. Schematic of a mouse brain showing the location of coronal sections C1 and C2 and RNA in situ hybridization showing expression of the Grp gene therein. Below is a diagram depicting the major areas that send auditory and contextual information to the amygdala obtained from tract-tracing studies.

FIG. 2: GRP Receptors Are Functionally Expressed in Interneurons of the Lateral Nucleus of the Amygdala.

(A1) Bath application of GRP (200 nM) increased frequency of sIPSCs in a pyramidal cell from a control mouse. The effect was blocked by 3 uM bombesin antagonist (n=6), thus suggesting that the GRP-induced enhancement of GABAergic tonic inhibition was specifically linked to the activation of the GRP receptors.

(A2) Effect of GRP on the frequency of sIPSCs is TTX-sensitive, and thus is dependent on action potential firing in interneurons.

(A3) GRP failed to increase the frequency of the picrotoxin-sensitive sIPSCs in GRPR knockout mice.

(B1) Representative sIPSCs recorded in a pyramidal cell from a control mouse at a holding potential of −70 mV under baseline conditions (left), during GRP application (center), and after the GRPR antagonist was added (right).

(B2) Representative sIPSCs recorded in a pyramidal neuron from GRPR knockout mouse under, baseline conditions (left), during GRP application (center), and after picrotoxin was added (right).

(C) Cumulative amplitude histograms of sIPSCs recorded under baseline conditions (filled symbols) and after GRP was applied (open symbols) in slices from control (left) and GRPR knockout mice.

FIG. 3: Pairing-Induced LTP is Enhanced in GRPR Knockout Mice. Pairing-induced LTP of whole-cell EPSCs recorded in the lateral amygdala in wild-type mice under control conditions (open symbols) and in the presence of the bombesin antagonist (3 uM, filled symbols).

(A) A schematic representation of a brain slice containing the amygdala that shows position of the recording and stimulation pipettes.

(B) LTP of whole-cell EPSCs recorded in the lateral amygdala neuron in response to the cortical input stimulation in slices from control (open symbols) or GRPR knockout (filled symbols) mice. For induction of LTP, the lateral amygdala neuron was held at +30 mV, and 80 presynaptic stimuli were delivered at 2 Hz to the external capsule fibers (arrow).

(C) Current-voltage plot of the GABA_{A} receptor IPSCs at holding potentials of −110 mV to −10 mV. Reversal potential of the IPSC mediated by the GABA_{A} receptors was −71 mV. Synaptic currents were recorded in the presence of the AMPA receptor antagonist CNQX (20 uM) and NMDA receptor antagonist D-APV (50 uM). Inset shows GABA_{A} receptor IPSCs recorded at holding potentials of −110 mV to −10 mV. Traces are averages of 10 IPSCs recorded at each holding potential.

(D) Pairing-induced LTP of whole-cell EPSCs recorded in the lateral amygdale in wild-type mice under control conditions (open symbols) and in the presence of the bombesin antagonist (3 uM, filled symbols).

FIG. 4: GRPR-Deficient Mice Have Enhanced and Resistant Long-Term But Not Short-Term Amygdala-Dependent Fear Memory

(A1) Contextual fear conditioning. Significant difference in freezing responses between GRPR knockout mice (n=9, solid bars) and wild-type (n=9, open bars) mice was found at 24 hr, 2, 7, and 15 weeks after training.

(A2) Cued fear conditioning. In response to the tone (CS), both groups showed an increase in freezing. However, this increase was significantly higher in GRPR knockout animals, although no difference was found between groups in the level of freezing before the onset of the tone (pre-CS).

(B1) Contextual and (B2) cued-fear conditioning assessed 30 min or 4 hr after training was normal in GRPR knockout mice.

(C1-4) Water Maze. (wild-type, n=9; knockout, n=9). In this hippocampus-dependent memory task, both groups of mice showed a similar rate of learning as demonstrated by their equivalent latency (C1) to reach the platform, whether it is during the visible (Day 1 and 2) or hidden platform version of the task (Day 3-6). They displayed the same swimming speed

(C2), and thigmotaxis (% of time spent at the periphery; C3). They also showed equivalent performance in the probe trial (% of time spent in the different quadrant areas; C4), which assessed the retention of spatially acquired information necessary to perform this task. GRPR knockout mice are no more sensitive or stressed than wild-type mice (D and E).

(D) Pain sensitivity thresholds. The intensity of shock required to elicit three reactions, movement (movt), vocalization (vocal), and jump, was assessed and data are presented as the mean±SEM. No difference was found between groups (wild-type, n=10; knockout, n=8).

(E) Elevated plus maze assessing basal anxiety. No difference was found between GRPR (n=18) and wild-type mice (n=16) in the total number of entries, as in the number of entries in the closed or open arms.

FIG. 5: A Model for GRP-Dependent Negative Feedback to Principal Neurons in the Amygdala in Wild-Type and GRPR Knockout Mice.

### DETAILED DESCRIPTION OF THE INVENTION

### Definitions

As used herein, “administering” shall mean delivering in a manner which is effected or performed using any of the various methods and delivery systems known to those skilled in the art. Administering can be performed, for example, intravenously, orally, via implant, transmucosally, transdermally, intramuscularly, or subcutaneously. “Administering” can also be performed, for example, once, a plurality of times, and/or over one or more extended periods.

As used herein, “agent” shall include, without limitation, an organic compound, a nucleic acid, a polypeptide, a lipid, and a carbohydrate. Agents include, for example, agents which are known with respect to structure and/or function, and those which are not known with respect to structure or function.

As used herein, “agonist of gastrin-releasing peptide receptor”, or its synonymous term “gastrin-releasing peptide receptor agonist”, shall mean an agent that, when bound to gastrin-releasing peptide receptor, stimulates a biological response like the biological response stimulated when gastrin-releasing peptide is bound to gastrin-releasing peptide receptor. For example, an agonist of the GRP receptor can enhance inhibitory function of interneurons containing GRPR, which leads to increased GABA release by interneurons. The magnitude of the biological response stimulated by a gastrin-releasing peptide receptor agonist can be the same as, greater than or less than the biological response stimulated by gastrin-releasing peptide. Methods of identifying gastrin-releasing peptide receptor agonists are well-known in the art. (see e.g. U.S. Pat. No. 5,741,651, at column 2, line 38). Gastrin-releasing peptide agonists include, without limitation, bombesin, gastrin-releasing peptide fragments, and mutants of gastrin-releasing peptide and its fragments (e.g. point mutants), and analogs of gastrin-releasing peptide and its fragments (e.g. gastrin-releasing peptide and its fragments wherein one or more amino acid residues are substituted with an amino acid derivative). Amino acid derivatives are well known in the art (see, e.g. U.S. Pat. No. 6,552,061). Additional agonists are described in Darker et al. 2001, and include, for example, GRP (aminoacids 19-27; available from Bachem, USA); [D-Phe⁶, βALa¹¹, Phe¹³, Nle¹⁴]Bn(6-14) amide; [Glp⁷, βAla¹¹, Phe¹³, Nle¹⁴]Bn(7-14) amide; [βALa¹¹, Phe¹³, Nle¹⁴]Bn(9-14) amide; and [βALa¹¹, Phe¹³, Nle¹⁴]Bn(10-14) amide. These agonists have very high affinity to GRPR, but very little or no affinity to other receptors from the mammalian bombesin family.

As used herein, a “fear-related disorder” shall mean any disorder induced by or resulting from an event that causes apprehension or alarm in the afflicted subject.

As used herein, “gastrin-releasing peptide” is a naturally occurring peptide that elicits gastrin release and regulates gastric acid secretion and motor function in a subject. Gastrin-releasing peptide can be from a human or any other subject. The terms “gastrin releasing peptide”, “gastrin-releasing peptide” and “GRP” are synonymous.

As used herein, with respect to claims for transgenic animals and methods of making same, “gastrin-releasing peptide gene”, or “GRP gene”, shall mean a naturally occurring GRP-encoding DNA sequence (including introns), contiguous at its 5′ end with at least about 30 kb of DNA sequence which is naturally contiguous with the 5′ end of the GRP-encoding DNA sequence, and contiguous at its 3′ end with at least 30 kb of the DNA sequence which is naturally contiguous with the 3′ end of the GRP-encoding DNA sequence.

As used herein, “inhibiting” the onset of a disorder shall mean either lessening the likelihood of the disorder's onset, or preventing the onset of the disorder entirely. In the preferred embodiment, inhibiting the onset of a disorder means preventing its onset entirely.

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

As used herein, “protein” and “polypeptide” are used equivalently, and each shall mean a polymer of amino acid residues. The amino acid residues can be naturally occurring or chemical analogues thereof.

Polypeptides and proteins can also include modifications such as glycosylation, lipid attachment, sulfation, hydroxylation, and ADP-ribosylation.

As used herein, “subject” shall mean a human or any animal, such as a non-human primate, mouse, rat, guinea pig, dog, cat, or rabbit.

As used herein, a “traumatic experience” includes, without limitation, military combat, physical assault, witnessing a physical assault, and experiencing a natural disaster, animal attack, or an emergency situation.

As used herein, “treating” a subject afflicted with a disorder shall mean either lessening the severity of the disorder, or eliminating the disorder entirely.

### EMBODIMENTS OF THE INVENTION

The present invention is based upon the discovery that gastrin-releasing peptide (GRP) is also an important regulator of certain types of anxiety, namely those involving amygdala-dependent learned fear.

Specifically, this invention provides a method for treating a subject afflicted with a fear-related disorder comprising administering to the subject a therapeutically effective amount of a gastrin-releasing peptide receptor agonist.

In one embodiment of the instant therapeutic method, the subject is human. Fear-related disorders treated include, for example, phobia, chronic anxiety, panic attack, post-traumatic stress disorder, and autism.

This invention further provides a method for inhibiting in a subject the onset of a fear-related disorder resulting from exposure to a traumatic experience comprising administering a prophylactically effective amount of a gastrin-releasing peptide receptor agonist to the subject prior to and/or following the traumatic experience.

In one embodiment of the instant prophylactic method, the subject is human. An agonist would be administered, for example, prior to a foreseeable traumatic experience such as military combat. In one embodiment, the agonist would be administered between 1 and 20 days, or 5 and 10 days prior to the traumatic experience. In a further embodiment, the agonist would be administered between 1 and 48 hours, or 12 and 24 hours prior to the traumatic experience. In a further embodiment, the agonist would be administered between 60 and 120 minutes, or 1 and 30 minutes prior to the traumatic experience.

An agonist would also be administered, for example, following a traumatic experience such as a physical assault. In one embodiment, the agonist would be administered between 1 and 20 days, or 5 and 10 days, following the traumatic experience. In a further embodiment, the agonist would be administered between 1 and 48 hours, or 12 and 24 hours following the traumatic experience. In a further embodiment, the agonist would be administered between 60 and 120 minutes, or 1 and 30 minutes following the traumatic experience.

Determining a therapeutically or prophylactically effective amount of the agonists used in the instant invention can be done based on animal data using routine computational methods. In one embodiment, the therapeutically or prophylactically effective amount contains between about 0.1 mg and about 1 g of agonist. In another embodiment, the effective amount contains between about 1 mg and about 100 mg of agonist. In a further embodiment, the effective amount contains between about 10 mg and about 50 mg. of agonist.

This invention further provides an article of manufacture comprising (a) a packaging material having therein a gastrin-releasing peptide receptor agonist, and (b) a label indicating a use for the agonist in treating, and/or inhibiting the onset of, a fear-related disorder in a subject.

This invention further provides a nucleic acid comprising a gastrin-releasing peptide gene, wherein the gene has inserted into it, either at its start or stop codon, a polypeptide-encoding sequence, wherein the polypeptide is not gastrin-releasing peptide. For mice, the GRP gene is located on chromosome 18. The gene contains 3 exons, a transcript length of 862 bp, and a translation length of 146 residues. All information regarding the GRP gene (coded by Q8R1I2 (SPTREMBL ID)) is contained at Ensemble site at http://www.ensembl.org/Mus_musculus/geneview?gene=ENSMUSG0 0000024517.

This invention further provides a transgenic animal whose somatic cells have stably integrated therein a nucleic acid comprising a gastrin-releasing peptide gene, wherein the gene has inserted into it, either at its start or stop codon, a polypeptide-encoding sequence, wherein the polypeptide is not gastrin-releasing peptide, and wherein the polypeptide is specifically expressed in the animal's amygdala. In one embodiment of the instant method, the transgenic animal is a mouse. Methods for making transgenic animals by introducing foreign DNA into embryonic cells and transplanting resulting cells into the uterus of an animal for development to term are well known in the art. (see e.g. U.S. Pat. No. 4,870,009). The foreign DNA can be introduced either by gene targeting “knock-in” technology or BAC transgenic technology (using DNA recombination in bacteria).

Finally, this invention provides a method for producing a transgenic animal whose amygdaloid cells specifically express an exogenous polypeptide, which method comprises producing a transgenic animal by introducing into an oocyte an exogenous DNA so that the exogenous DNA is stably integrated into the oocyte, and permitting the resulting oocyte to mature into a viable animal, wherein (a) the animal's somatic cells have the exogenous DNA stably integrated therein, (b) the exogenous DNA comprises a gastrin-releasing peptide gene, wherein the gene has inserted into it, either at its start or stop codon, an exogenous polypeptide-encoding sequence, and the exogenous polypeptide is not gastrin-releasing peptide, and (c) the exogenous polypeptide is specifically expressed in the animal's amygdala.

This invention is illustrated in the Experimental Details section which follows. This section is set forth to aid in an understanding of the invention but is not intended to, and should not be construed to, limit in any way the invention as set forth in the claims which follow thereafter.

### EXPERIMENTAL DETAILS

### Synopsis

The present invention identifies two genes highly enriched in the lateral nucleus of the amygdala: the gastrin-releasing peptide (GRP) and oncoprotein 18 (Op18)/Stathmin. We focused on GRP because it is presumably released as a cotransmitter with glutamate in pyramidal cells of the lateral nucleus and its receptor (GRPR) has been pinpointed as a candidate in autism. This invention demonstrates that, when released by activity, GRP acts on and excites inhibitory interneurons by activating GRPR on their cell surface. Activation of GRPR in these interneurons in turn leads to an increase in the level of tonic GABAergic inhibition in the principal neurons. This invention further demonstrates that in amygdala slices from GRPR knockout mice, the tonic inhibition is markedly reduced and LTP is enhanced. Consistent with this finding, these mice have enhanced and prolonged long-term memory for fear to both auditory and contextual cues, suggesting that the GRP signaling pathways serves as an inhibitory feedback constraint on learned fear.

### METHODS

### Animals

GRPR knockout mice were described before and were found grossly normal (Hampton et al., 1998). Mice used for the study were back-crossed to N10 or more to C57BL/6J strain.

### Differential Screening

Amygdala cells were acutely dissociated as described (Yu and Shin-nick-Gallagher, 1997). Cells morphologically resembling pyramidal neurons were identified under low magnification Nikon microscope and individually transferred to PCR tubes containing lysis buffer. cDNA libraries were synthesized as described (Dulac and Axel, 1995). Five thousand clones were differentially screened with the amygdala and CA1 single cell cDNA probes. Amygdala probes for the differential screening were enriched by two rounds of subtraction of representational difference analysis (Hubank and Schatz, 1994) against the CA1 cDNA.

### In Situ Hybridization/Immunohistochemistry

Coronal sections from fresh-frozen mouse brains were cut 20 microns thick and hybridized according to the published protocol with modifications (Schaeren-Wiemers and Gerfin-Moser, 1993). For dual fluorescent in situ hybridization and immunohistochemistry, digoxigenin-labeled RNA was first detected using tyramide-based TSA Direct Fluorescein Kit (Perkin Elmer). Then, sections were incubated with rabbit antibody recognizing glutamic acid decarboxylase (Chemicon) and detected using Cy3-conjugated donkey anti-rabbit IgG (Jackson ImmunoResearch).

### Electrophysiology

Amygdala slices (250-300 um) were prepared from 3-5 week old control and GRPR knockout mice (littermates) with a vibratome. Slices were continuously superfused in solution containing (in mM): 119 NaCl, 2.5 KCl, 2.5 CaCl₂,1.0 MgSO₄, 1.25 NaH₂PO₄, 26.0 NaHCO₃, 10 glucose, and equilibrated with 95% O₂ and 5% CO₂ [pH 7.3-7.4] at room temperature. Whole-cell recordings of evoked compound EPSCs or spontaneous GABA-mediated IPSCs were obtained from pyramidal cells in the lateral amygdala under visual guidance (DIG/ infrared optics) with an EPC-9 amplifier and Pulse v8.09 software (HEKA Elektronik). Compound EPSCs were evoked by stimulation of the fibers in the external capsule at 0.05 Hz with a concentric stimulating electrode consisting of a patch pipette (10 um tip diameter) that was coated with silver paint (Bolshakov et al., 1997). The two leads of the stimulus isolation unit (ISO-Flex, Master-8 stimulator, AMPI, Jerusalem, Israel) were connected to the inside of the pipette and the external silver coat. The stimulating pipette was positioned to activate the cortical input to the lateral amygdala. To elicit the evoked GABA_{A} IPSCs in the presence of CNQX (20 uM) and D-APV (50 uM) in the bath, the stimulation electrode was placed within the lateral nucleus of the amygdala. The patch electrodes (3-5 MH resistance) contained (in mM): 120 KCl, 5 NaCl, 1 MgCl₂, 0.2 EGTA, 10 HEPES, 2 MgATP, and 0.1 NaGTP (adjusted to pH 7.2 with KOH). In LTP experiments, 120 mM K-gluconate was used instead of KCI. To examine the voltage dependence of the evoked GABA_{A} receptor IPSCs, cesium was substituted for potassium in the pipette solution. Series resistance was monitored throughout experiment and was in a range of 10-20 milliohlms. Currents were filtered at 1 kHz and digitized at 5 kHz. The holding potential was −70 mV. In all LTP experiments, the stimulus intensity was adjusted to produce synaptic responses with an amplitude which constitutes ˜20%-25% of maximum amplitude EPSC. Since we controlled for the size of the baseline EPSC, the induction conditions were identical for both LTP groups (control and knockout mice). The EPSC amplitudes were measured as the difference between the mean current during a prestimulus baseline and the mean current over a 2 ms-window at the peak of the response. For induction of LTP, 80 presynaptic stimuli were delivered at 2 Hz to the external capsule fibers while the lateral nucleus of the amygdala neuron was held at +30 mV for the duration of the LTP-inducing presynaptic stimulation. Summary LTP graphs were constructed by normalizing data in 60 s epochs to the mean value of the baseline EPSC.

The spontaneous IPSCs were recorded on videotape for off line analysis in the presence of 20 uM CNQX. Data were analyzed with the Mini Analysis Program v5.2.4 (Synaptosoft Inc., Decatur, Ga.; Bolshakov et al., 2000).

### Behavior

For all behavioral tasks, mutant and control littermates (males, 3 months old) were used. Statistical analyses used ANOVAs with genotype as the between subject factor, and session (fear conditioning experiment), day, area (quadrant or platform in the Morris water maze), or zone (elevated plus maze and light-dark box) as within subject factors. Mean±SEM are presented. The experimenter was blind to the genotype in all studies.

Fear conditioning experiments were done as described (Bourt-chouladze et al., 1998) On the training day, the mouse was placed in the conditioning chamber (Med Associates) for 2 min before the onset of CS, a tone, which lasted for 30 s at 2800 Hz, 85 dB. The last 2 s of the CS was paired with US, 0.7 mA of continuous foot shock. After an additional 30 s in the chamber, the mouse was returned to its home cage. Conditioning was assessed for 3 consecutive min in the chamber in which the mice were trained by scoring freezing behavior, which was defined as the complete lack of movement, In intervals of 5 s. Mice (wild-type, n=9; knockout, n=9) were tested immediately after training and at 24 hr, 2, 7, and 15 weeks after training. For each time point, testing occurred first in the context in which mice were trained (contextual fear conditioning). Three hours after each contextual testing session, mice were placed in a novel environment (cued fear conditioning) in which the tone (120 s) that has been presented during training was given after a 1 min habituation period (pre-CS).

### Pain Sensitivity Tests

Response to foot shocks was assessed with naive mice (wild-type, n=10; knockout, n=8) as described (Harrel, 2001). The intensity of shock required to elicit running, vocalization, and a jump was determined for each mouse by delivering a 1 s shock every 30 s starting at 0.08 mA and increasing the shock 0.02 mA each time. Testing was stopped after all behaviors had been noted.

### Anxiety Tests

We performed two different tasks to assess basal anxiety level in naive mice.

### Elevated Plus Maze

The elevated plus maze consisted of a center platform and four arms placed 50 cm above the floor (Ramboz et al., 1998). Two arms were enclosed within walls and the other two (open) had low rims. Naive mice (wild-type, n=18; knockout, n=16) were placed in the center and their behavior was recorded for 5 min with a camera located above the maze. Time spent (in seconds, s) and entries in the different compartments (closed and open arms, center) were assessed.

### Dark-Light Box

For the dark-light box test, mice (wild-type, n=10; knockout, n=9) were placed in the dark compartment (head facing the wall) and observed for 5 min (Johansson et al., 2001). Time spent in and entries into the lit compartment were recorded.

### Water Maze

The task was performed as previously described (Malleret et al., 1999) with two training phases: 2 days with a visible platform followed by 4 days (spatial phase) with a hidden platform in the training quadrant (wild-type, n=9; knockout, n=9). For each phase, four trials, 120 s maximum and 15 min ITI (intertrial interval) were given daily. Probe trials (60 s), during which the platform was removed, were performed to assess retention of the previously acquired information.

### RESULTS

### Isolation of Genes Specifically Expressed in the Lateral Nucleus of the Amygdala

As an initial step in characterizing the molecular mechanisms involved in learned fear, we searched for genes enriched in the amygdala and, in particular, in the lateral nucleus. To this end, we focused on pyramidal projection neurons because these cells form the majority of the constituent neurons in the cortex-like nuclei of the amygdala to which the lateral nucleus belongs and they transmit the CS and US information during fear learning. We isolated neurons using acute dissociation, which preserves their processes and allows cell identification based on neuronal morphology under the microscope (Yu and Shinnick-Gallagher, 1997). Similarly, pyramidal neurons were isolated from the anterior dorsal CA1 subregion of the hippocampus, which was chosen for the comparison during cDNA library screening because this region may be less involved in learned fear as opposed to the ventral hippocampus (Bast et al., 2001). We first used two rounds of representation difference analysis (RDA) to enrich the lateral nucleus CDNA probe against the CA1 CDNA sequences. After differential screening of CDNA library derived from single pyramidal amygdala neuron with probes from the lateral nucleus and the CA1 region, we analyzed candidate clones for gene expression pattern using RNA in situ hybridization. We found two genes, Grp and Oncoprotein 18 (Op18)/Stathmin expressed in the lateral nucleus of the amygdala that had low or no expression in the CA1 region of the hippocampus. Interestingly, these two genes are also expressed in the accessory basal nucleus (AB) of the amygdala, but are absent in the basal lateral nucleus (BLA) that is located between the lateral nucleus of the amygdala and AB.

Both the Grp and Op18/Stathmin sequences originated from the screening of the same cell, which we identified as glutamatergic pyramidal neuron based on its shape during acute dissociation under the microscope and later by hybridizing its cDNA library with different neuronal and glial markers and by subsequent characterization of the sequences comprising this cDNA library. This cDNA library (that contained the Grp and Op18/Stathmin sequences) was positive for neurofilament-L (NF-L, neuronal marker) and it was negative for glial fibrilary acidic protein (GFAP, glial marker) and glutamic acid decarboxilase (GAD, interneuronal marker). In addition, we isolated from this library a cDNA that corresponds to the zinc transporter-3 (ZnT-3) gene, a specific marker for zinc-containing subgroup of glutamatergic neurons, highly enriched in the limbic system and the lateral nucleus of the amygdala.

### GRP is Expressed in the Lateral Nucleus of the Amygdala and in the Regions Sending Synaptic Projections to the Lateral Nucleus

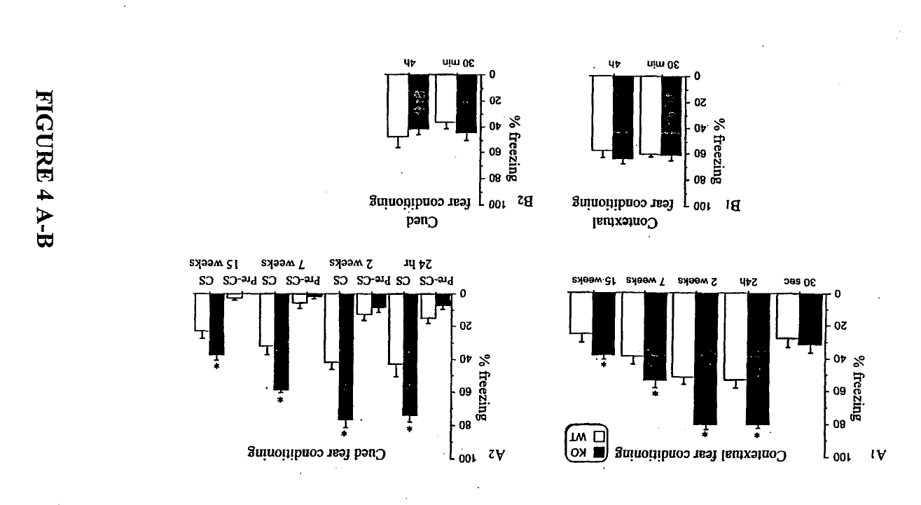

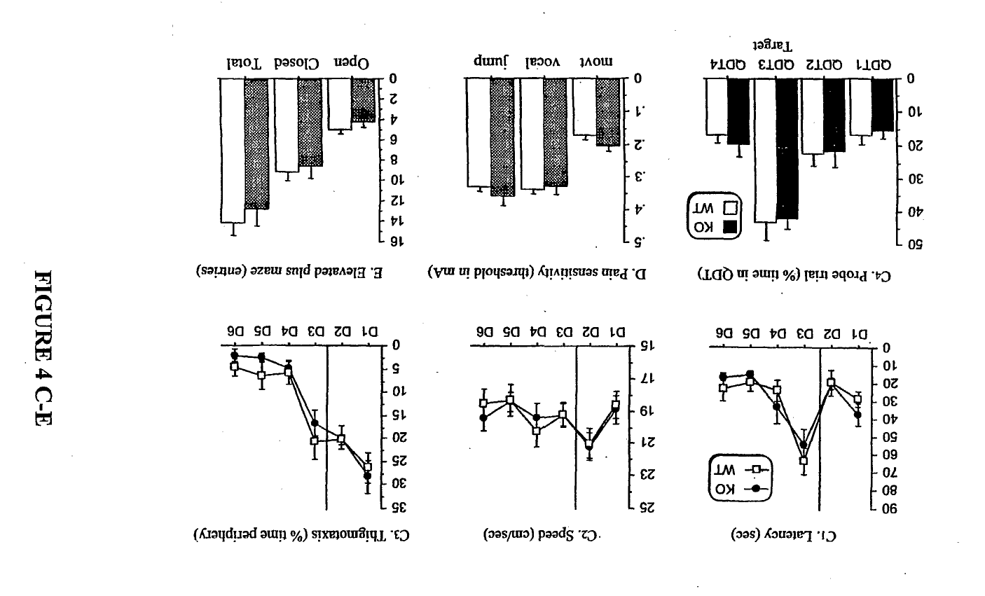

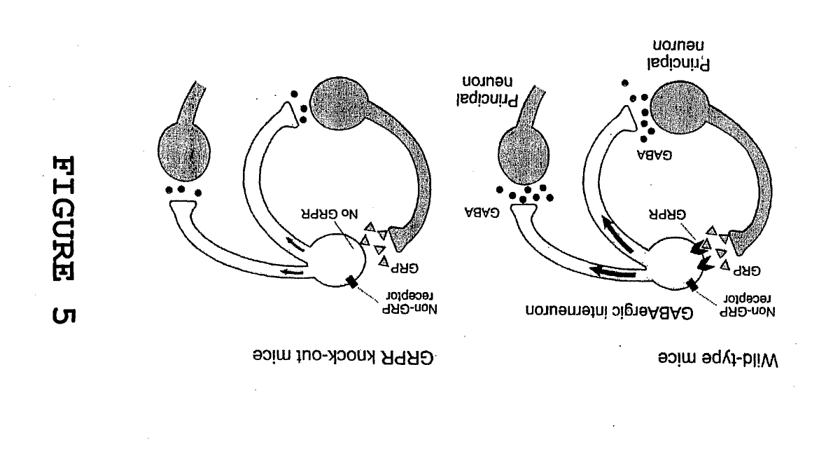

Using in situ hybridization, we next found that the Grp gene is highly enriched in the lateral nucleus of the amygdala, and more specifically, in its dorsal and medial subnuclei. In addition, we observed strong expression in the medial, ventral, and dorsal subdivisions of the medial geniculate body (MGm, MGv, and MGd), the posterior intralaminar nucleus (PIN) of the auditory thalamus, the TE3 subregion of the auditory cortex, and the perirhinal cortex (PRh, FIG. 2B). All of these regions are afferently connected with the lateral nucleus of the amygdala and provide auditory inputs to the lateral nucleus of the amygdala during fear learning (Pitkanen et al., 1997) suggesting that this peptide is involved in auditory cued fear conditioning. For example, MGm and PIN directly project auditory information to the lateral nucleus of the amygdala and to TE3. Area T3 of the cortex in turn projects to the lateral nucleus of the amygdala (LeDoux, 2000). The ventral subiculum (VS), another structure where the Grp is localized, also provides a strong input to the medial division of the lateral nucleus of the amygdala as well as to BLA and AB. PRh is reciprocally connected with the lateral nucleus of the amygdala and is capable of sending either cued or contextual signals. GRP is also expressed in the ventral dentate gyrus. However, a connection between the lateral nucleus of the amygdala or AB and the dentate gyrus is not well documented.

### GRPR is Expressed in Inhibitory-Interneurons

GRP is a peptide neurotransmitter that is selectively recognized by a seven transmembrane domain receptor (GRPR) coupled to Gaq-protein (Hellmich et al., 1999). Having shown that GRP is expressed by principal cells in the lateral nucleus of the amygdala, we were curious to know what types of cells express GRPR. To identify the neurons within the lateral nucleus of the amygdala that express GRPR, we performed colocalization studies using dual fluorescent in situ hybridization for Grpr RNA and immunohistochemistry with antibodies against interneuron-specific marker, glutamic acid decarboxylase (GAD67 form, FIG. 3A) . We found that the Grpr RNA was expressed selectively in inhibitory GABAergic interneurons. However, GRPR was present only in a subpopulation of GAD-positive interneurons, which suggests that the lateral nucleus of the amygdala contains various groups of interneurons subserving different functions.

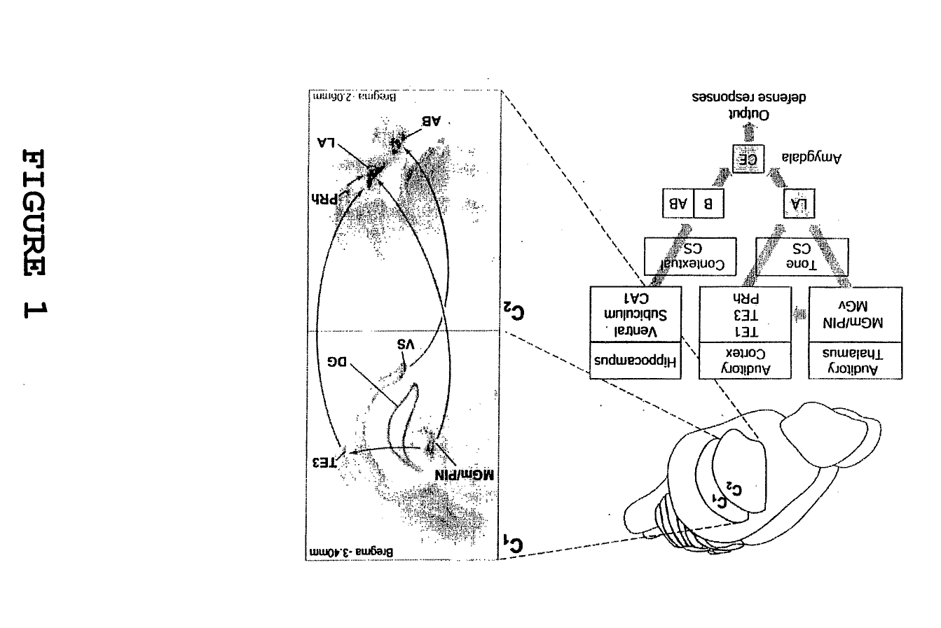

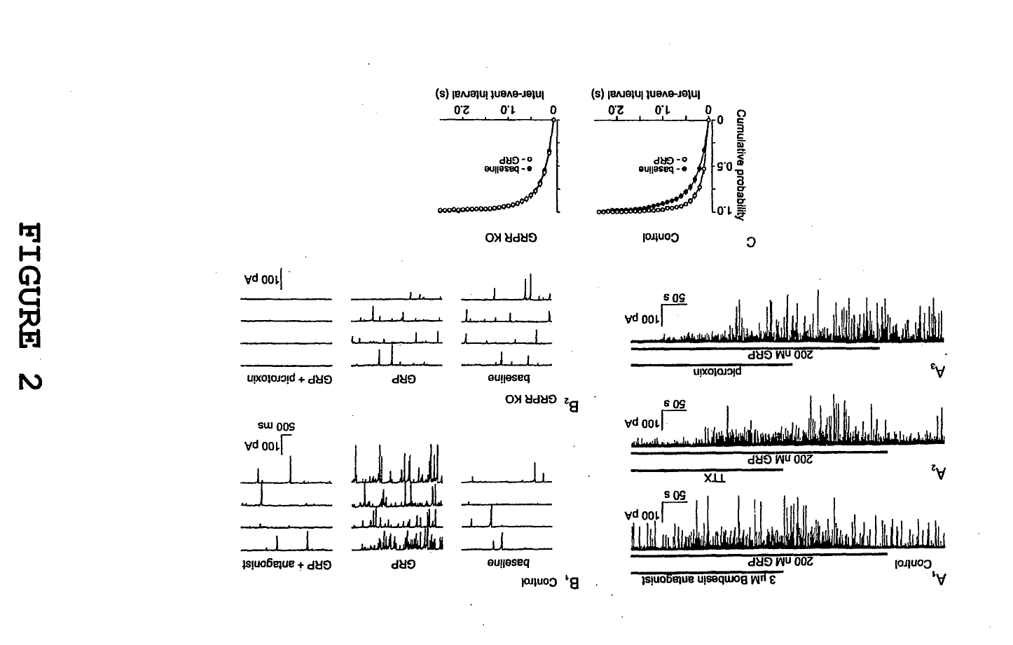

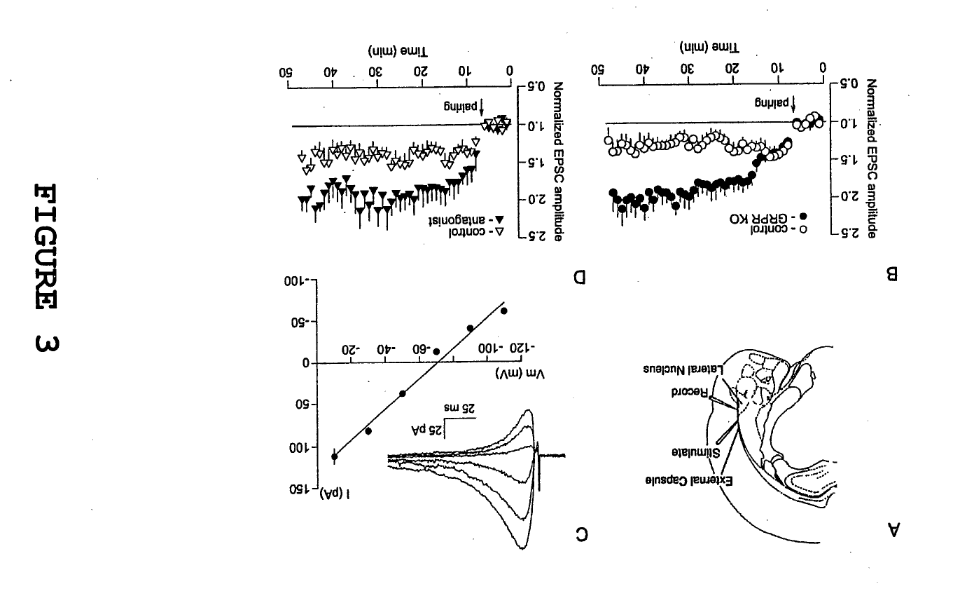

Physiological, tract-tracing, and immunocytochemical studies have shown that afferent signals converging onto the lateral nucleus of the amygdala are regulated locally in the dorsolateral division by inhibitory interneurons (Woodson et al., 2000). The afferent glutamatergic projections to the amygdala synapse on both principal cells and GABAergic inhibitory interneurons (Mahanty and Sah, 1998). The inhibitory interneurons in turn send feedback inhibitory projections to pyramidal neurons. These feedback and feedforward GABAergic inputs are thought to determine how the excitatory inputs to the principal cells involved in fear learning are processed and conveyed along neural pathways in the amygdala (Wang et al., 2001). The observed pattern of the Grp and Grpr genes expression suggested to us that GRPR exerts a functional role in modulating the balance between excitation and inhibition in the local neuronal networks related to learned fear (see FIG. 1).

### GRP Appears to Excite GABAergic Inhibitory Interneurons in the Lateral Nucleus of the Amygdala that Functionally Express GRPR

To test whether activation of the GRP receptors on the GABAergic interneurons in the lateral nucleus of the amygdala by the release of GRP from principal cells can change the level of tonic inhibition in the principal cell, we carried out whole-cell recordings from visually identified pyramidal neurons in mice. We identified pyramidal neurons based on their appearance and their ability to demonstrate spike frequency adaptation to the prolonged depolarizing current injection (Tsvetkov et al., 2002).

We recorded spontaneous inhibitory postsynaptic currents (sIPSCs) in the pyramidal neurons having blocked the AM PA (a-amino-3-hydroxy-5-methyl-4-isoxazolepropionic acid) receptor-mediated responses (FIG. 2) with CNQX (6-cyano-7-nitroquinoxaline-2,3-dione, 20 uM). To increase the inhibitory signals, we inverted the inhibitory currents so that they had an inward direction by dialyzing the postsynaptic cells with a chloride-based intrapipette solution. Consistent with the notion that the sIPSCs are mediated by the GABA_{A} receptors, these currents were completely blocked (FIGS. 2A₃ and 2B₂) by gamma-aminobutyric acid-A (GABA_{A}) receptor antagonist, picrotoxin (50 uM, n=10), at a holding potential of −70 mV.

In the absence of a GABA_{A} receptor blockade, application of GRP (200 nM) led to a significant increase in the frequency of sIPSCs in the soma of the principal cells of wild-type mice (baseline: 5.23±0.68 Hz; GRP: 10.12±1.0 Hz; n=17 cells, obtained from 5 control mice; significant difference, paired t test, t=4.99, P<0.0002; FIGS. 2A-2C). Therefore, we think that the increase in frequency of GABA sIPSCs was likely due to excitation of the interneurons by GRP leading to an increase in the firing of action potentials in GABAergic interneurons. We further supported this by blocking the effects of the agonist by applying a Na⁺ channel blocker tetrodotoxin (TTX, 1 uM, n=7; FIG. 2A₂) These findings in the lateral amygdala are consistent with previous work in the hippocampus, where bombesin-like neuropeptides (including GRP) elicited a marked increase in the frequency of GABA_{A} receptor-mediated IPSCs recorded in CA1 pyramidal neurons (Lee et al., 1999) mediated by depolarization and induced repetitive firing of GABAergic interneurons in the stratum oriens.

We specifically linked the observed effect of the bath-applied GRP to the activation of GRPR. Bath application of a specific antagonist of GRPR ([D-Phe⁶,Des-Met¹⁴]-bombesin-(6-14)ethyl amide; 3 uM; Lee et al., 1999) blocked the effect of GRP on the frequency of sIPSCs (FIGS. 2A, and 2B,; baseline: 5.15±0.91 Hz; GRP: 10.37±1.2 Hz; antagonist of GRPR: 5.72±1.1 Hz; n=6 cells) . The difference in the frequency of sIPSCs in the baseline conditions and after the GRPR antagonist application was not statistically significant (paired t test, t=1.21, P=0.3), suggesting that the bombesin antagonist fully abolished the GRP-induced increase in the frequency of the sIPSCs.

### Knockout of GRPR Eliminates Tonic Inhibition

To obtain independent evidence that GRP induces enhancement of GABAergic tonic inhibition due to activation of GRP receptors localized on interneurons, we turned to mice in which the gene for GRPR was knocked out. These mutant mice were littermates of the control mice we have studied to this point. GRPR knockout mice do not show any obvious developmental anatomical abnormalities throughout their body or their brain (Hampton et al., 1998 and FIG. 3B). Immunohistochemistry on brain sections of these mice with interneuron-specific antibodies (pan/albumin, calretinin, and calbindin) revealed no differences between knockout mice and wild-type controls. However, in situ hybridization revealed that the GABAergic interneurons in the knockout mice were lacking GRPR. Consistent with these findings, we found in the mutants that the GRP-mediated negative control of the excitatory synaptic inputs to principal cells in the lateral nucleus was lacking. In slices from mice in which the Grpr gene was ablated, bath-applied GRP failed to increase the frequency of sIPSCs (200 nM; baseline: 5.06±0.58 Hz; GRP: 5.64±0.67 Hz; n−23 cells, obtained from 6 GRPR knockout mice; no significant difference: paired t test, t=1.04, P=0.31; FIGS. 4A₃, 4B₂, and 4C). These results suggest that GRP receptors are functionally expressed in the lateral nucleus of wild-type mice and that activation of the GRP receptors on these interneurons was responsible for the dramatic increase in the level of tonic GABA inhibition observed in the principal neurons in the lateral nucleus.

### LTP in the Cortico-Amygdala Pathway is Enhanced in GRPR-Knockout Mice

Our recent findings indicate that LTP of the synaptic connections in the neural circuit of learned fear is an essential cellular mechanism contributing to the acquisition of memory for fear (Tsvetkov et al. 2002; see also McKernan and Shinnick-Gallagher, 1997; Rogan et al., 1997). Studies of different brain regions, including the hippocampus (Steele and Mauk, 1999), the cortex (Trepel and Racine, 2000), and the amygdala (Rammes et al., 2000), indicate that modulation of principal cells by GABA-mediated inhibition can play an important role in the induction of LTP. We therefore asked: does removal of GRPR in the inhibitory interneurons affect LTP, in slices of the lateral nucleus of the amygdala?

We induced LTP of the compound glutamatergic EPSCs at the cortico-amygdala synapses by pairing postsynaptic depolarization from a holding potential of −70 mV to +30 mV with 80 presynaptic stimuli delivered to the fibers in the external capsule (Huang and Kandel, 1998; Mahanty and Sah, 1998; Weisskopf and LeDoux, 1999) at a frequency of 2 Hz (FIGS. 3A-3B). We measured LTP with the K-gluconate containing intrapipette solution, without picrotoxin in the bath (see Experimental Procedures). Under these experimental conditions, the peak amplitude of the evoked EPSC was solely determined by activation of the AMPA glutamate receptors. The contribution of the GABA_{A} receptor-mediated component to the EPSC was negligible at such holding potential since it was very close to the reversal potential (Eᵣ) of GABA_{A} IPSC (−67±3 mV, n=6; FIG. 3C). This induction protocol was used because, as we have shown previously, it consistently produces robust LTP (Tsvetkov et al., 2002). We have deliberately chosen to depolarize a postsynaptic cell to a more positive membrane potential during the induction period, than in some previous studies, to allow a maximal activation of L-type Ca²+ channels (e.g., Mermelstein et al., 2000), as they were shown to take part in the induction process (Tsvetkov et al., 2002; Weisskopf et al., 1999). Keeping Ca²⁺ influx through L-type Ca²⁺ channels at a relatively constant level, we minimize a possible non-linearity of the interaction between the NMDA receptor and Ca²⁺ channel-mediated contribution to the integral postsynaptic calcium signal, thus maintaining the more uniform induction conditions. When LTP at the cortical input to the amygdala was compared (in a blinded fashion) in slices from control and from GRPR knockout mice, we found that LTP was significantly greater in knockout than in control mice (FIG. 3B), with an average LTP of the EPSC to 2.02±0.2 (n=12 cells) and 1.33±0.13 (n=9 cells) of the baseline EPSC value, respectively. The difference in the amount of LTP measured over a 5 min period (between 35 and 40 min after pairing) between control and knockout mice was statistically significant (t-test, t=2.96, P<0.01). Thus, the ablation of the Grpr gene disinhibits the pyramidal cells and makes the cortico-amygdala synapses more susceptible to LTP. To obtain independent support for this conclusion, we measured the pairing-induced LTP in slices from wild-type mice in the presence of the bombesin antagonist. Under these conditions, LTP also was significantly enhanced (FIG. 5D, control LTP: 1.42±0.04, n=5 cells; LTP with the antagonist: 1.92±0.05, n=7 cells; significant difference, t test, t=8.1, P<0.0001).

### GRPR-Deficient Mice have Enhanced and Persistent Long-Term Memory for Fear to Both Auditory and Contextual Cues

We first trained GRPR-deficient mice in Pavlovian cued and contextual fear conditioning, an amygdala-dependent task, which depends on the ability of the animal to learn and remember that auditory cue or context predict electric shock. During training, the level of overall freezing of knockout animals was not significantly different from wild-type littermate controls For both groups, freezing was slightly increased within 30 s immediately after training (FIG. 4A) . When tested for amygdala-dependent tone fear conditioning, mice were placed in a new context 24 hr after training (FIG. 4A₂) . Mice displayed an increase in freezing at the onset of the tone (CS; cued fear conditioning) as compared to the freezing prior (pre-CS) to the tone (Session effect, all p<0.01). In addition, the ANOVA revealed a significant effect of genotype showing that GRPR knockout mice froze more than the wild-type mice at the presentation of the tone which had been associated previously with the electric shock (genotype effect: [F(1,16)=13.30; p−0.002]). Although freezing decreased with time in both groups of mice (Session effect; all p<0.01), GRPR knockout mice produced a higher response to the tone in subsequent CS cued-testing sessions at 2, 7, and 15 weeks (Genotype effect; all p<0.05).

Contextual fear conditioning is dependent both on the amygdala and the hippocampus. Here, mice were tested in the absence of cue in the same context 24 hr after training. Both mutant and wild-type mice exhibited higher level of freezing compared to immediately after the shock (Session effect, all p<0.0001, FIG. 4A. This suggests that the mutant mice not only remembered the context where they received the shock the day before, but that they also developed with time a strong aversive response to this environment associated with a painful experience. The ANOVA revealed a significant effect of genotype ([F(1,16)=25.07; p=0.0001]) showing that both groups of mice froze differently in this context, with GRPR knockout mice showing a higher response as compared to their control littermates. Although freezing to context decreased in both groups of mice with time (Session effect, all p<0.0001) suggesting similar rate of extinction, the observed increase in freezing in GRPR knockout mice was still present in subsequent testing sessions at 2, 7, and 15 weeks (Genotype effect, all p<0.05).

We also analyzed mutant mice for short-term memory at 30 min and at 4 hr in independent groups. For both time points, there was no significant difference between mutant and wild-type mice in both contextual and cued fear conditioning (FIG. 4B) . Thus, the enhancement in learned fear observed in GRPR knockout mice is specific to long-term but not short-term memory.

To verify that the increase in freezing displayed by GRPR knockout mice in the fear conditioning experiment was not due to an increased sensitivity to the shock, we performed a control experiment in which we administered electric shock of increasing intensity while recording the behavioral response exhibited by the mice (Harrel, 2001). There was no difference between groups in the intensity of shock required to elicit movement, vocalization, or jump (FIG. 2D), indicating that an increase in freezing observed in the fear conditioning experiments was due to the learning process and not to a difference in pain sensitivity.

### The Enhanced and Persistent Fear is Learned and not Secondary to Chronic Anxiety

To explore further these mice's tendency for innate (not learned) fear, we used the elevated plus maze where mice face a conflict between an innate aversion to the open arms of the maze and the motivation to explore this compartment (Ramboz et al., 1998). The ANOVA conducted on the number of entries in the open and closed arms and on the index of anxiety (time spent/ entries in the open arms) did not reveal any significant effect of genotype (FIG. 4E) . Thus, in the elevated plus maze, the basal level of anxiety was similar in control and GRPR knockout mice.

Another way to assess anxiety in mice is a light-dark box test (Johansson et al., 2001). In this test, mice tend to avoid the light compartment and naturally prefer the dark one. Here again, we did not find any difference between groups in the number of entries as well as the total time spent in the lit compartment. Thus, as with the elevated plus maze, the results from the light-dark box test suggest that the basal level of anxiety in GRPR knockout mice is similar to that of wild-type mice.

### The GRPR Knockout Mice Show Normal Hippocampus-Dependent Spatial Memory

Because GRP is expressed in the lateral nucleus of the amygdala and specifically in its circuitry for learned fear and we have found that knockout of GRPR enhances amygdala-based learning, we were curious to know if we can use GRPR-deficient mice to dissociate amygdala-dependent from hippocampus-dependent learning. To determine whether GRPR is important for a purely hippocampus-based task, we turned to the Morris water maze, a task in which the amygdala is not involved. In this maze, an animal has to remember the position of a hidden escape platform in relationship to distal cues surrounding it in a circular pool (Malleret et al., 1999). During acquisition of the Morris water maze, mice from all groups showed a decrease in escape latency (FIG. 4C₁) across days, indicating learning of the platform position (all groups, p<0.0001). They also showed a preference for the target quadrant during the probe trial performed on the last day of the experiment (FIG. 4C₄) . We found no differences between groups in this task (no genotype effect), suggesting that the deletion of the GRPR does not enhance hippocampus-dependent learning that is independent of the amygdala, which is similar to the results of Wada and coworkers (Wada et al., 1997). These results support the notion that the amygdala is directly involved in learned fear (Fanselow and LeDoux, 1999) and that it does not merely modulate memories formed in other brain structures like the hippocampus.

### DISCUSSION

We have identified, characterized, and localized to a specific inhibitory neural circuit in the lateral nucleus of the amygdala a molecular signaling network important for learned fear. When this inhibitory molecular network is disrupted, mice show increased LTP in the lateral nucleus of the amygdala and an enhanced memory of learned fear as evident in both cued and contextual fear conditioning. There is a normal memory for hippocampus-based spatial task indicating that this network is specifically involved in the regulation of memory formation in the amygdala in response to danger signals. There also is no alteration in innate fear.

Experiments in humans and in experimental animals over the last half a century indicate that the amygdala is involved in learned fear (Davis and Whalen, 2001; LeDoux, 2000). In the past 50 years, we have learned a fair amount about the anatomy and cell physiology underlying amygdala-based fear. For example, recent experiments have demonstrated that the mechanisms of LTP are recruited behaviorally at the synapses in the lateral nucleus of the amygdala during training for learned fear (Rogan et al., 1997; McKernan and Shinnick-Gallagher, 1997; Tsvetkov et al., 2002), thus providing direct support for the link between LTP and memory storage. By contrast, very little is known about the molecular mechanisms contributing to this form of fear. This is unfortunate because the neuronal pathways carrying sensory information for unimodal learned fear (the information carried by the CS) is much better specified than that for the sensory information for spatial learning as is the correlation between LTP and memory storage.

We therefore have isolated amygdala-enriched genes and then, using mouse genetics in combination with physiological and behavioral approaches studied the role of these genes in the memory for fear.

Initially, we isolated two genes expressed in a glutamatergic principal neuron of the lateral nucleus of the amygdala. The first of these genes, Op18/Stathmin, is highly expressed both in the lateral nucleus of the amygdala and in the cerebral cortex with very little expression in the hippocampus. Op18/Stathmin is a phosphoprotein that binds tubulin dimers and destabilizes cellular microtubules (Belmont and Mitchison, 1996). It is a major substrate for protein kinase A and upon phosphorylation releases tubulin thus allowing polymerization of tubulin molecules. Op18/Stathmin mRNA levels are increased after lesions to the perforant pathway of the hippocampus, which together with the biochemical role of Op18/Stathmin protein suggests its involvement in synaptogenesis (Brauer et al., 2001).

The second gene, Grp, is uniquely localized in the lateral and accessory basal nuclei of the amygdala and in regions that send projections to it and which are essential for delivering information about CS to the amygdala during Pavlovian fear conditioning (LeDoux, 2000). In particular, our analysis showed that the Grp gene is expressed both in the areas specific to pathways delivering tone CS information and in the areas specific to pathways delivering contextual CS information. GRP is a 29 amino acid long mammalian homolog of the amphibian peptide bombesin (Kroog et al., 1995) and may serve as a cotransmitter with glutamate in pyramidal neurons in the rodent brain (Lee et al., 1999 and our present data) . Our observation of the Grp gene expression pattern specific to the fear network of the amygdala finds support in the report that GRP concentration was increased in the central nucleus of the rat amygdala during both stress and feeding (Merali et al., 1998). GRPR is a G q protein-coupled receptor and its downstream targets include protein kinase C (PKC-p) and phospholipase C as shown both in cultured mouse fibroblasts and rat hippocampal neurons (Mellmich et al., 1999; Lee et al., 1999). GRPR activation by GRP binding leads to intracellular release of Ca²⁺ and eventually to the activation of the MAPK pathway (Sharif et al., 1997).

We found that GRP is expressed in a group of glutamatergic principal neurons enriched in zinc. Interestingly, zinc-containing glutamatergic neurons constitute a specific network circuitry that includes the lateral nucleus of the amygdala and other components of the limbic system (reviewed in Frederickson et al., 2000). We next found, as did Lee and colleagues (1999), that GRPR is expressed in GABAergic interneurons. We also found that GRPR activation can significantly enhance the level of tonic GABA-mediated inhibition in the lateral nucleus. Recent pharmacological and genetic studies have shown that the establishment of a balance between glutamatergic excitatory and GABAergic inhibitory functions is critical for processing of information in the amygdala (Bast et al., 2001; Krezel et al., 2001). Based on these published data and our results, we suggest a model of GRP action in the amygdala during fear response; during excitation, the glutamatergic principal cells may release as a cotransmitter the excitatory peptide, GRP. Through the binding to GRPR on interneurons, GRP leads to GABA release. This may provide tonic, feedforward, or feedback inhibitory control of the processing of CS stimuli by principal cells (FIG. 7, left image). Thus, this molecular signaling pathway provides a control which can regulate the balance between excitatory and inhibitory circuitries in the amygdala.

### GRPR-Deficient Mice Show Both Enhanced LTP and Enhanced Memory Storage in Amygdala-Dependent Tasks

To test this model, we next examined the pyramidal neurons in the lateral nucleus of the amygdala of GRPR knockout animals and found that indeed they lack an inhibitory control normally provided by GRP in wild-type conditions (FIG. 7, right image). As a result of lacking inhibition, there is an enhanced LTP in the cortico-amygdala pathway. In agreement with our genetic finding, previous pharmacological work has demonstrated that modulation of the level of GABA-mediated inhibition of the principal cells in the amygdala may determine how easily LTP is induced at the amygdala synapses (Krezel et al., 2001; Rammes et al., 2000).

Consistent with an enhancement of LTP, these animals also show enhanced freezing in both cued and contextual versions of amygdala-dependent fear conditioning task. Throughout all time points tested (the latest 15 weeks after training), mutant mice had higher freezing than normal mice. This may be due to faster fear memory retrieval in mutant mice because during testing mutants started freezing right after the tone was turned on but wild-types froze a few seconds later. The fading over time of the phenotype of GRPR knockout mice for fear conditioning might reflect the contribution of shock-induced sensitization in addition to the enhancement in learning. In contrast to long-term effects, we found that mutant mice have normal short-term memory when tested at 30 min and even 4 hr after training. This finding suggests the interesting possibility that GRP/GRPR signaling pathway modulates learned fear in a long-term specific manner and thus provides further support to the notion that LTP is implicated in the mechanisms of long-term memory. Importantly, GRPR-deficient mice showed normal memory in the Morris water maze, which is dependent on the hippocampus but not the amygdala. This finding is again consistent with fear circuitry-specific expression pattern of the Grp gene and allowed us to dissociate amygdala-based behavior from hippocampus-based behavior. Thus, we identified a network that is specifically involved in amygdala-dependent long-term memories for fear.

### A Possible Role of GRP Pathway in Mental Disorders

The analysis of mice with decreased GABA function may have important clinical implications. Decreased levels of GABA have consistently been found in patients with depression, panic, and generalized anxiety disorders (Goddard et al., 2001) and some of the drugs currently used to treat panic and generalized anxiety disorders increase levels of GABA in the brain (Parent et al., 2002). We did not find any abnormalities in basal or in innate anxiety of GRPR knockout mice probably because we did not disrupt directly the biochemical machinery involved in GABA production and utilization. Rather, we interfered with the GABA functions by disrupting a network that regulates GABA release. The reduction in GABA release in mutant mice seems to fine-tune the memory storage system so as to improve memory storage for fear. Perhaps, greater depleting GABA would lead to the opposite effects; it might decrease memory storage for fear and lead to high levels of anxiety similar to that described in mice mutant for GABA receptors (Low et al., 2000; McKernan et al., 2000). Since of all mental disorders anxiety disorders are those that can best be modeled in mice and other experimental animals (Bachevalier et al., 2001), it is likely that molecular insights in the biology of fear will prove to be broadly informative regarding the genes important both for normal human fear and for anxiety states.

Indeed, recent studies have suggested the possible involvement of GRP and GRPR in mental disorders. GRPR is a candidate gene for autism; an X;8 translocation has been found that disrupted the first intron of the GRPR gene in an autistic female patient (Ishikawa-Brush et al., 1997). Importantly, genetic studies in autistic patients have pinpointed chromosomal abnormalities in the 15q11-q13, a region where the GABRB3 gene is located, which codes for the beta 3 subunit of the gamma-amino-butyric acid (GABA)_{A} receptor (Cook et al., 1998). Moreover, recent behavioral, anatomical, and neuroimaging studies suggest that one of the critical loci for autism resides in the amygdala (Baron-Cohen et al., 2000).

Taken altogether, these observations demonstrate the importance of determining molecular substrates of a mygdala-dependent memory processes and identify the components of GRP/GRPR molecular network as a clear target for treating anxiety disorders.

### REFERENCES

Bachevalier, J. et al. (2001). Effects of selective neonatal temporal lobe lesions on socioemotional behavior in infant rhesus monkeys (Macaca mulatta) Behav. Neurosci. 775, 545-559.
Baron-Cohen, S. et al. (2000). The amygdala theory of autism. Neurosci. Biobehav. Rev. 24, 355-364.
Bast, T. et al. (2001). The ventral hippocampus and fear conditioning in rats. Different anterograde amnesias of fear after tetrodotoxin inactivation and infusion of the GABA(A) agonist muscimol. Exp. Brain Res. 739, 39-52.
Belmont, L. D., and Mitchison, T. J. (1996). Identification of a protein that interacts with tubulin dimers and increases the catastrophe rate of microtubules. Cell 84, 623-631.
Bolshakov, V. Y. et al. (1997). Recruitment of new sites of synaptic transmission during the cAMP-dependent late phase of LTP at CA3-CA1 synapses in the hippocampus. Neuron 79, 635-651.
Bolshakov, V. Y. et al. (2000). Dual MAP kinase pathways mediate opposing forms of long-term plasticity at CA3-CA1 synapses. Nat. Neurosci. 3,1107-1112.
Bourtchouladze, R. et al. (1998). Different training procedures recruit either one or two critical periods for contextual memory consolidation, each of which requires protein synthesis and PKA. Learn. Mem. 5, 365-374.
Brauer, A. U. et al. (2001). Perforant path lesion induces up-regulation of stathmin messenger RNA, but not SCG10 messenger RNA, in the adult rat hippocampus. Neuroscience 702, 515-526.
Cook, E. H. et al. (1998). Linkage-disequilibrium mapping of autistic disorder, with 15q11-13 markers. Am. J. Hum. Genet. 62,1077-1083.
Darker J G et al. (2001). Discovery of potent and selective peptide agonists at the GRP-preferring bombesin receptor (BB2). J. Pept. Sci. 7(11):598-605.
Davis, M., and Whalen, P J. (2001). The amygdala: vigilance and emotion. Mol. Psychiatry 6,13-34.
Dulac, C., and Axel, R. (1995). A novel family of genes encoding putative pheromone receptors in mammals. Cell 83,195-206.
Fanselow, M. S., and LeDoux, J. E. (1999). Why we think plasticity underlying Pavlovian fear conditioning occurs in the basolateral amygdala. Neuron 23, 229-232.
Frederickson, C. J. et al. (2000). Importance of zinc in the central nervous system: the zinc-containing neuron. J. Nutr. 730,1471S-1483S.
Goddard, A. W. et al. (2001). Reductions in occipital cortex GABA levels in panic disorder detected with 1h-magnetic resonance spectroscopy. Arch. Gen. Psychiatry 58, 556-561.
Hampton, L. L. et al. (1998). Loss of bombesin-induced feeding suppression in gas-trin-releasing peptide receptor-deficient mice. Proc. Natl. Acad. Sci. USA 95, 3188-3192.
Harrel, A. V. et al. (2001). Transgenic mice over-expressing the 5-HT3 receptor have enhanced learning in latent inhibition and contextual fear conditioning paradigms. Paper presented at Abstracts, Society for Neuroscience, 31st Annual Meeting, San Diego, Calif.
Hellmich, M. R. (1999). Multiple protein kinase pathways are involved in gastrin-releasing peptide receptor-regulated secretion. J. Biol. Chem. 274, 23901-23909.
Huang, Y. Y., and Kandel, E. R. (1998). Postsynaptic induction and PKA-dependent expression of LTP in the lateral amygdala. Neuron 27,169-178.
Hubank, M., and Schatz, D. G. (1994). Identifying differences in mRNA expression by representational difference analysis of cDNA. Nucleic Acids Res. 22, 5640-5648.
Ishikawa-Brush, Y. et al. (1997). Autism and multiple exostoses associated with an X;8 translocation occurring within the GRPR gene and 3′ to the SDC2 gene. Hum. Mol. Genet. 6,1241-1250.
Johansson, B. et al. (2001). Hyperalgesia, anxiety, and decreased hypoxic neuroprotection in mice lacking the adenosine Al receptor. Proc. Natl. Acad. Sci. USA 98, 9407-9412.
Kapp, B. S. et al. (1992). Amygdaloid contributions to conditioned arousal and sensory information processing. In The Amygdala: Neurobiological Aspects of Emotion, Memory, and Mental Dysfunction, J. P. Aggleton, ed. (New York: Wiley-Liss), pp. 229-254.
Krezel, W. et al. (2001). Increased anxiety and synaptic plasticity in estrogen receptor p-deficient mice. Proc. Natl. Acad. Sci. USA 98,12278-12282.
Kroog, G. S. et al. (1995). Mammalian bombesin receptors. Med. Res. Rev. 75, 389-417.
LeDoux, J. E. (2000). Emotion circuits in the brain. Annu. Rev. Neurosci. 23, 155-184.
Lee, K. et al. (1999). Bombesin-like peptides depolarize rat hippocampal interneurones through interaction with subtype 2 bombesin receptors. J. Physiol. 578, 791-802.
Low, K. et al. (2000). Molecular and neuronal substrate for the selective attenuation of anxiety. Science 290,131-134.
Mahanty, N. K., and Sah, P. (1998). Calcium-permeable AMPA receptors mediate long-term potentiation in interneurons in the amygdala. Nature 94, 683-687.
Malleret, G. et al. (1999). 5-HT1B receptor knock-out mice exhibit increased exploratory activity and enhanced spatial memory performance in the Morris water maze. J. Neurosci. 79, 6157-6168.
McKernan, M. G., and Shinnick-Gallagher, P. (1997). Fear conditioning induces a lasting potentiation of synaptic currents in vitro. Nature 390, 607-611.
McKernan, R. M. et al. (2000). Sedative but not anxiolytic properties of benzodiazepines are mediated by the GABA(A) receptor alpha subtype. Nat. Neurosci. 3, 587-592.
Merali, Z. et al. (1998). Aversive and appetitive events evoke the release of corticotropin-releasing hormone and bombesin-like peptides at the central nucleus of the amygdala. J. Neurosci. 78, 4758-4766.
Mermelstein, P. G., et al. (2000). Critical dependence of cAMP response element-binding protein phosphorylation on L-type calcium channels supports a selective response to EPSPs, in preference to action potentials. J. Neurosci. 20, 266-273.
Parent, M. B. et al. (2002). Effects of the antidepressant/antipanic drug phenelzine and its putative metabolite phenylethylidenehydrazine on extracellular gamma-aminobutyric acid levels in the striatum. Biochem. Pharmacol. 63, 57-64.
Pitkanen, A. et al. (1997). Organization of intra-amygdaloid circuitries in the rat: an emerging framework for understanding functions of the amygdala. Trends Neurosci. 20, 517-523.
Ramboz, S. et al. (1998). Serotonin receptor 1A knockout: an animal model of anxiety-related disorder. Proc. Natl. Acad. Sci. USA 95, 14476-14481.
Rammes, G. et al. (2000). Synaptic plasticity in the basolateral amygdala in transgenic mice expressing dominant-negative cAMP response element-binding protein (CREB) in forebrain. Eur. J. Neurosci. 72, 2534-2546.
Rogan, M. T. et al. (1997). Fear conditioning induces associative long-term potentiation in the amygdala. Nature 390, 604-607.
Romanski, L. M., and LeDoux, J. E. (1992). Equipotentiality of thalamoamygdala and thalamocortico-amygdala circuits in auditory fear conditioning. J. Neurosci. 72, 4501-4509.
Schaeren-Wiemers, N., and Gerfin-Moser, A. (1993). A single protocol to detect transcripts of various types and expression levels in neural tissue and cultured cells: in situ hybridization using digoxigenin-labeled cRNA probes. Histochemistry 700, 431-440.
Sharif, T. R. et al. (1997). Functional expression of bombesin receptor in most adult and pediatric human glioblas-toma cell lines; role in mitogenesis and in stimulating the mitogen-activated protein kinase pathway. Mot. Cell. Endocrinol. 130, 119-130.
Steele, P. M., and Mauk, M. D. (1999). Inhibitory control of LTP and LTD: stability of synapse strength. J. Neurophysiol. 81, 1559-1566.
Trepel, C., and Racine, R. J. (2000). GABAergic modulation of neocortical long-term potentiation in the freely moving rat. Synapse 35, 120-128.
Tsvetkov, E. et al. (2002). Fear conditioning occludes LTP-induced presynap-tic enhancement of synaptic transmission in the cortical pathway to the lateral amygdala. Neuron 34, 289-300.
Wada, E. et al. (1997). Generation and characterization of mice lacking gastrin-releasing peptide receptor. Biochem. Biophys. Res. Commun. 239, 28-33.
Wang, C. et al. (2001). Role of NMDA, non-NMDA, and GABA receptors in signal propagation in the amygdala formation. J. Neurophysiol. 86, 1422-1429.
Weisskopf, M. G., and LeDoux, J. E. (1999). Distinct populations of NMDA receptors at subcortical and cortical inputs to principal cells of the lateral amygdala. J. Neurophysiol. 81, 930-934.
Weisskopf, M. G. et al. (1999). L-type voltage-gated calcium channels mediate NMDA-independent associative long-term potentiation at thalamic input synapses to the amygdala. J. Neurosci. 79, 10512-10519.
Woodson, W. et al. (2000). Afferents from the auditory thalamus synapse on inhibitory interneurons in the lateral nucleus of the amygdala. Synapse 38, 124-137.
Yu, B., and Shinnick-Gallagher, P. (1997). Dihydropyridine- and neurotoxin-sensitive and insensitive calcium currents in acutely dissociated neurons of the rat central amygdala. J. Neurophysiol. 77, 690-701.

What is claimed is:

1. A method for treating a subject afflicted with a fear-related disorder comprising administering to the subject a therapeutically effective amount of a gastrin-releasing peptide receptor agonist.

2. The method of claim 1, wherein the subject is human.

3. The method of claim 1, wherein the fear-related disorder is a phobia.

4. The method of claim 1, wherein the fear-related disorder is chronic anxiety.

5. The method of claim 1, wherein the fear-related disorder is a panic attack.

6. The method of claim 1, wherein the fear-related disorder is post-traumatic stress disorder.

7. The method of claim 1, wherein the fear-related disorder is autism.

8. A method for inhibiting in a subject the onset of a fear-related disorder resulting from exposure to a traumatic experience comprising administering a prophylactically effective amount of a gastrin-releasing peptide receptor agonist to the subject prior to and/or following the traumatic experience.

9. The method of claim 8, wherein the subject is human.

10. The method of claim 8, wherein the fear-related disorder is a phobia.

11. (canceled)

12. The method of claim 8, wherein the fear-related disorder is a panic attack.

13. The method of claim 8, wherein the fear-related disorder is post-traumatic stress disorder.

14. The method of claim 8, wherein the agonist is administered to the subject prior to the traumatic experience.

15. The method of claim 14, wherein the traumatic experience is military combat.

16. The method of claim 8, wherein the agonist is administered to the subject after the traumatic experience.

17. The method of claim 16, wherein the traumatic experience is a physical assault.

18. (canceled)

19. A nucleic acid comprising a gastrin-releasing peptide gene, wherein the gene has inserted into it, either at its start or stop codon, a polypeptide-encoding sequence, wherein the polypeptide is not gastrin-releasing peptide.

20. A bacterial artificial chromosome (BAC) comprising the nucleic acid of claim 19.

21.-24. (canceled)

25. An article of manufacture comprising (a) a packaging material having therein a gastrin-releasing peptide receptor agonist, and (b) a label indicating a use for the agonist in treating, and/or inhibiting the onset of, a fear-related disorder in a subject.

26. A transgenic animal whose somatic cells have stably integrated therein a nucleic acid comprising a gastrin-releasing peptide gene, wherein the gene has inserted into it, either at its start or stop codon, a polypeptide-encoding sequence, wherein the polypeptide is not gastrin-releasing peptide, and wherein the polypeptide is specifically expressed in the animal's amygdale.

27. A method for producing a transgenic animal whose amygdaloid cells specifically expresss an exogenous poypeptide, which method comprises producing a transgenic animal by introducing into an oocyte an exogenous DNA so that the exogenous DNA is stably integrated into the oocyte, and permitting the resulting oocyte to mature into a viable animal, wherein (a) the animal's somatic cells have the exogenous DNA stably integrated therein, (b) the exogenous DNA comprises a gastrin-releasing peptide gene, wherein the gene has inserted into it, either at its start or stop codon, an exogenous polypeptide is not gastrin-releasing peptide, and (c) the exogenous polypeptide is specifically expressed in the animal's amygdale.
